(19) United States
(12) Patent Application Publication
(10) Pub. No.: US 2011/0144476 A1
(43) Pub. Date: Jun. 16, 2011

(54) Integrated Surgical Sampling Probe

(75) Inventors: Ferenc Jolesz, Brookline, MA (US); Alexandra Golby, Boston, MA (US); Nathalie Agar, Newton, MA (US)

(73) Assignee: THE BRIGHAM AND WOMEN'S HOSPITAL, INC., Boston, MA (US)

(21) Appl. No.: 13/059,524

(22) PCT Filed: Aug. 17, 2009
(86) PCT No.: PCT/US09/53967

§ 371 Date: Feb. 17, 2011

(30) Foreign Application Priority Data
Aug. 18, 2008 (US) 61/136190

(51) Int. Cl. A61B 10/02; A61B 5/055; A61B 6/03

(52) U.S. Cl. 600/411; 600/566; 600/424; 600/427

(57) ABSTRACT

The present invention is directed to devices for collecting tissue samples from patients. The devices create tissue fragments which are then collected by aspiration. The devices include signal emitters that allow the exact position where a biopsy sample was obtained to be determined. In addition the invention includes methods for obtaining tissue samples using these devices.

### FIELD OF THE INVENTION

The present invention is directed to medical devices that can be used to obtain tissue samples for diagnostic evaluation and which are integrated with a stereotactic coordinate system so that the exact location of biopsy sites can be determined. In addition, the invention includes methods of collecting tissue samples using these devices.

### BACKGROUND OF THE INVENTION

Stereotactic surgical procedures were developed in the early 1900's and were first applied clinically in the 1940's (Kelly, P., Neurosurgery 46:16 (2000)). Initially these procedures were used in neurosurgery and involved affixing an external apparatus to a patient's skull to establish a coordinate system for locating, in a reproducible manner, the exact position of a lesion within the intracranial area. Today, stereotactic procedures have been applied to other tissues and are typically used in conjunction with diagnostic imaging procedures such as CT scans and MRIs to map internal tissues, prior to, or during, surgery (see, e.g., Poza, et al., Appl. Neurophysiol., 48:482-487 (1985); Dorwald, et al. Br. J. Neurosurg. 16:110-118 (2002); Krieger, et al., J. Surg. Oncol. 14:13-25 (1998)).

The development of stereotactic methods and imaging techniques has been accompanied by the development of surgical instruments that allow physicians to perform procedures at sites that were formerly inaccessible. Among the most successful of the instruments that have been developed for neurosurgery are probes designed to ultrasonically ablate tissue. For example, the Cavitron Ultrasonic Surgical Aspirator® (Integra Radionics) uses pulses of ultrasonic energy delivered to a needle-like tip to fragment tissue, which is concurrently irrigated and removed by aspiration. Although probes of this type were initially designed primarily for the surgical resection of tumors, it was subsequently found that the tissue fragments generated by the devices maintain sufficient integrity to be used diagnostically (Richmond, et al., Neurosurg. 13:415-419 (1983); Malhotra, et al., Acta Neurochir. 81:132-134 (1986); Blackie, et al., J. Clin. Pathol. 37:1101-1104 (2008)).

In addition to probes that ablate tissue ultrasonically, probes such as the Nico Myriad™ probe (NICO Corporation) have been designed to perform surgical ablations by mechanically cutting or shaving tissue. One attractive aspect of these “mechanical sampling” probes is that tissue is obtained without the generation of heat.

Despite the advances noted above, the diagnostic use of ultrasonic and mechanical probes has gone largely undeveloped and potential advantages over traditional methods of tissue sampling have often gone unrecognized.

### SUMMARY OF THE INVENTION

The present invention is based upon the concept of integrating a tissue resection device with a stereotactic navigation system and then using the device to collect tissue fragments for diagnostic assays. This allows tissue sampling locations to be precisely determined. Preliminary results and published articles reporting on the histopathological evaluation of tissue fragments indicate that ultrasonically generated fragments preserve the features required for standard histopathological diagnosis. It is expected that mechanically generated fragments would also preserve these features.

In its first aspect, the invention is directed to a medical device (also referred to herein as a “probe”) that can be used in collecting tissue samples from biopsy sites in a patient. Examples of devices that may be adapted for this purpose can be found in U.S. Pat. Nos. 4,827,911; 4,861,332; 4,660,573; 4,320,761; 4,561,438; 4,169,984; 4,223,676; 4,425,115; and 7,137,963, all of which are incorporated by reference herein in their entirety. The device includes a hand held support, typically made of plastic, metal or rubber with a shape and size that allows it to be easily held and maneuvered in an operator's hand. Typically, these supports will have a rectangular or cylindrical shape and be about 4 to 8 inches in length, although other shapes and sizes are possible. Extending from, and attached to, one end of the hand held support is an elongated metal rod with a proximal end (the end attached to the support) and a distal end (the end furthest from the support). The rod will typically be about 3 to 10 inches long and terminate at its distal end in a tip that either itself vibrates in response to ultrasonic energy or which has a separate component attached to it that vibrates in response to ultrasonic energy. Alternatively the tip may include a sharpened cutting surface that, in response to electrical stimulation, cuts or shaves tissue.

The medical device also includes means for supplying ultrasonic energy to the tip or to the separate vibrational component, preferably at a frequency of 15-100 kHz and, more preferably, at 20-60 kHz. Systems for generating ultrasonic vibrations have been known in the art for many years and have been used in many types of devices (see e.g., U.S. Pat. Nos. 2,594,841; 3,975,650; 4,736,130; 4,868,445; 5,076,854; 5,276,376; and 5,496,411, all of which are incorporated herein by reference in their entirety). Alternatively the device may be designed to respond to the input of electrical energy by moving in a manner that results in the cutting or shaving of tissue. For example, there may be an electrical motor that causes the tip to rotate in the manner of a drill in response to electrical input.

In addition, the device includes means for supplying irrigating fluid to the distal end of the tip and for aspirating tissue fragments created at the tip as the result of ultrasonic vibrations (see e.g., U.S. Pat. Nos. 3,693,613; 4,063,557 and 4,827,911, all of which are incorporated by reference herein in their entirety) or due to mechanical cutting or shaving. A preferred method for supplying irrigating fluid is by pumping it from a reservoir through a tubular channel running through the rod and terminating in an opening at the tip. The exact diameter of the channel is not critical to the invention but will typically be between ⅛ and ½ of an inch. The reservoir may contain any pharmaceutically acceptable fluid such as water, saline, Ringer's solution etc. and may be maintained at room temperature or chilled, e.g., to 0-15° C. If desired, the fluid may also include antibiotics to help prevent infection or other drugs.

With respect to aspiration, it is preferred that the metal rod of the device have a hollow core that provides a fluid passageway for tissue fragments. This passageway is open at the tip and extends through or past the hand held support of the device. Sufficient suction is provided, e.g., by means of a medical suction pump, to aspirate material through the opening at the tip in the direction of the hand held support. As with the channel for providing irrigation fluid, the diameter of the passageway for aspiration is not critical but will, in general, be between ⅛ and ½ inch.

The passageway should be connected at its proximal end to a tissue collection container where aspirated fragments are delivered and which, in some embodiments, contains a fluid such as water, saline or Ringer's solution. This fluid may, optionally be chilled, e.g., to 0-10° C., and may include chemicals for fixing tissue samples. In an alternative embodiment, the tissue fragments may be delivered to a container in which they are quick frozen, e.g., in dry ice or liquid nitrogen. Examples of irrigation and aspiration systems may be found in U.S. Pat. Nos. 6,723,110; 6,565,535; 4,504,264; 4,531,934; and 4,637,814 (all of which are incorporated by reference herein in their entirety) as well as in references cited above.

A primary improvement with respect to the present medical device is that it is integrated with a system for stereotactically determining the position of the distal end of the rod (i.e., the location where aspirated tissue samples are collected) relative to the tissue being examined (e.g., brain tissue). Any of the stereotactic positioning systems that are known in the art may be adapted for this purpose. Examples of such systems and related materials are described in U.S. Pat. Nos. 4,618,978; 5,171,296; 5,207,688; 5,311,868; 5,383,454; 5,423,832; 5,531,229; 5,628,315; 5,665,095; 5,690,108; 5,706,811; 5,728,106; 5,947,981; and 6,355,049 (all of which are incorporated by reference herein in their entirety).

The stereotactic system should include a computer that stores information regarding the spatial relationship between the probe (particularly the tip of the probe) and the tissue of the patient being examined. The probe includes means for communicating information to the computer regarding its position. This may be accomplished using, inter alia: a) ultrasound detectors (see e.g., U.S. Pat. Nos. 7,251,352; 7,142,905, incorporated by reference herein in their entirety); b) electromagnetic emitters located on the device (preferably on the hand held support of the device) that transmit signals to a separate electromagnetic receiver (see U.S. Pat. Nos. 7,357,573; 7,313,430, incorporated by reference herein in their entirety); c) sound emitters located on the device (preferably on the hand held support) that transmit signals to microphones (see U.S. Pat. No. 6,374,135, incorporated by reference herein in its entirety); or d) by “optical tracking using infrared energy detectors (see U.S. Pat. No. 7,302,288, incorporated by reference herein in its entirety). In each instance, signals are communicated to the computer for analysis. The most preferred method for communicating information concerning the position of the device is with electromagnetic emitters.

In a preferred embodiment, the hand held support includes an actuator switch which, when activated, permits the transmission of ultrasonic or mechanical energy to the tip of the rod or to a separate component which vibrates in response to ultrasonic energy. When the switch is not activated ultrasonic energy is not transmitted. Activation of the actuator switch is, preferably, accompanied by the transmission of a signal to the computer to aid in determining the position of the tip of the rod at the time of actuation. In an alternative and more preferable design, the actuator switch is in the form of a foot pedal which, when activated, transmits ultrasonic energy to the tip of the rod. Actuator switches may also be used which, instead of causing rod tips to ultrasonically vibrate, cause the tip to move in a manner that results in the cutting or shaving of tissue.

In another aspect, the invention is directed to a method of collecting a tissue sample from a biopsy site by inserting the tip of any of the medical devices described above into a patient so that the distal end of the rod is positioned at the site where the biopsy is to be performed. Energy is then transmitted to the tip of the rod to create ultrasonic vibrations at the site and fragment tissue or to cause the cutting or shaving of tissue. Irrigation fluid is then administered at the biopsy site and the fragments are aspirated into a collection container where they are retrieved for histological examination or other diagnostic tests.

The invention includes methods of collecting tissue samples that are mapped to a particular biopsy site. The first step in these methods is to establish a three dimensional stereotactic coordinate system for reproducibly identifying positions in the tissue that is to be examined, e.g., a portion of a patient's brain or a tumorous growth. Any of the stereotactic positioning systems described in the various references cited above can be used for this purpose. Next, one or more diagnostic imaging procedures (e.g., a CT or MRI scan) are performed to identify areas in the patient where one or more biopsy samples may be taken. Finally, tissue samples are collected using a medical device that fragments the tissue, collects the fragments that have been generated and records the position of sampling in the stereotactic coordinate system. The information obtained using this procedure will be particularly useful when multiple sites are sampled, for example, to determine how far cancer cells have invaded. Although the methodology can, in principle be applied to any site in a patient's body, it is expected that, initially, it will be most useful for biopsies involving brain tissue or breast tissue.

### BRIEF DESCRIPTION OF THE DRAWINGS

FIG. 1: FIG. 1 illustrates a type of probe that may be adapted for use in the present invention. The probe disrupts cellular tissue through longitudinal vibration of a hollow tip at ultrasonic frequencies (24 or 35 kHz). Combining the disruption process with irrigation coming from the annular space surrounding the probe assists in removal of the tissue by aspiration through the center of the handpiece into a waste container. For the purposes of the present invention, the waste container is replaced with a collection container and/or analytical device. The figure shows two electromagnetic sensors (J) that have been located on the hand held support of the device for signaling its position.

FIG. 2: FIG. 2 is a drawing of a device that has a hand held support (K) and an elongated metal rod (L).

FIG. 3: FIG. 3 is an illustration of a hand held support for a device showing a channel for the flow of irrigation fluid (B); and an opening (D) leading into a passageway for aspirated tissue fragments (I). A reservoir of irrigation fluid is represented as (A) and feeds into the channel B through port (G). The aspirated tissue exits the passageway carrying aspirated tissue (I) in a stream (E) passing through port (H) and may be collected in any suitable specimen container. At the distal end of the hand held support is a coupling region (C) where the elongated rod may be attached. In the embodiment shown, attachment is accomplished using threaded bore (M). The figure also shows (F) which represents a cord for transmitting energy that causes the tip of the device to vibrate or move in a manner that results in the cutting or shaving of tissue.

FIG. 4: FIG. 4 shows the terminal part of a device that includes an elongated metal rod L terminating in an opening (N), through which tissue samples may be aspirated. In the embodiment shown, the rod is designed to attach to the hand held support by means of a threaded regions at its base (O) that can be screwed into a matching region in the hand held support (M in FIG. 3).

### DETAILED DESCRIPTION OF THE INVENTION

The present invention is directed to systems for obtaining tissue samples that record the exact location where each sample is taken. Existing devices that have previously been used primarily for tissue ablation may be adapted for the retrieval of tissue samples and modified so that they integrate with existing systems for the stereotactic positioning of medical devices.

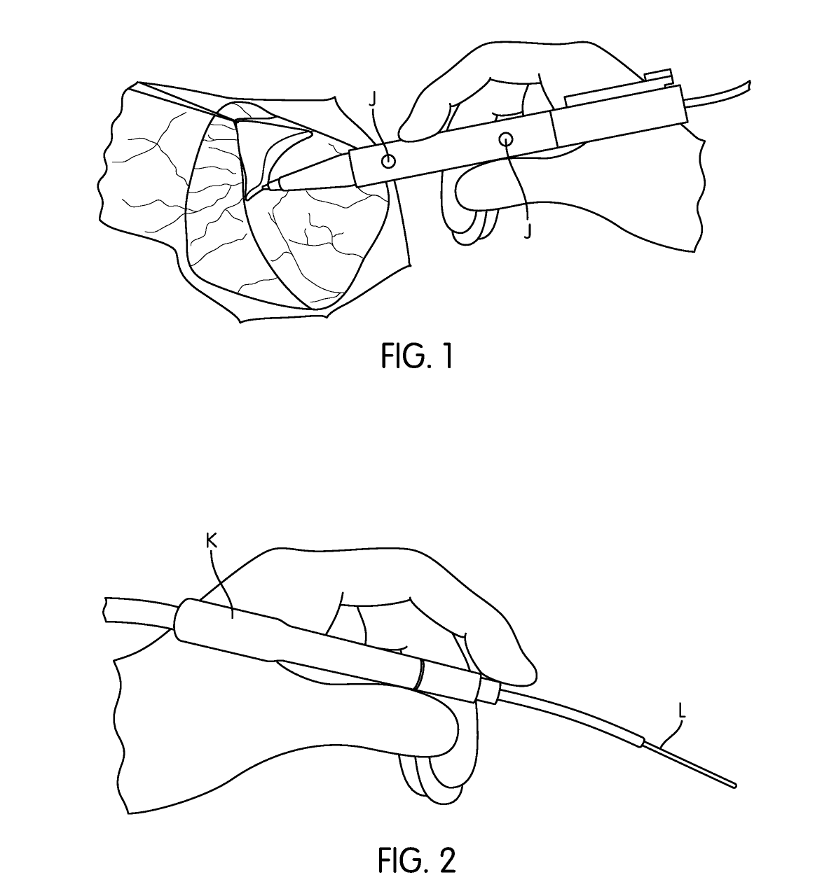

Devices that utilize ultrasonic vibrations to fragment tissues and that recover the fragments by aspiration are one type of device that is particularly well suited to the invention because fragments produced in this manner have been shown to retain histological characteristics that can be used diagnostically. An example of such a device is the Cavitron Ultrasonic Surgical Aspirator (CUSA), made by Integra Radionics and similar to the device illustrated in FIG. 2. The hand held base unit of the device is shown as (K) with a curved and elongated metal rod (L) extending from it. Devices of this type can be modified by incorporating a component into the hand held support that will provide a signal that can be used in analyzing its exact position. For example, electromagnetic emitters may be included in the support to provide a signal to a separate receiver, which, in turn, communicates this information to a computer for analysis. A drawing of a device with electromagnetic emitters (J) is shown in FIG. 1. Other signaling systems that may be used include those that detect ultrasonic signals, sound signals and infrared signals.

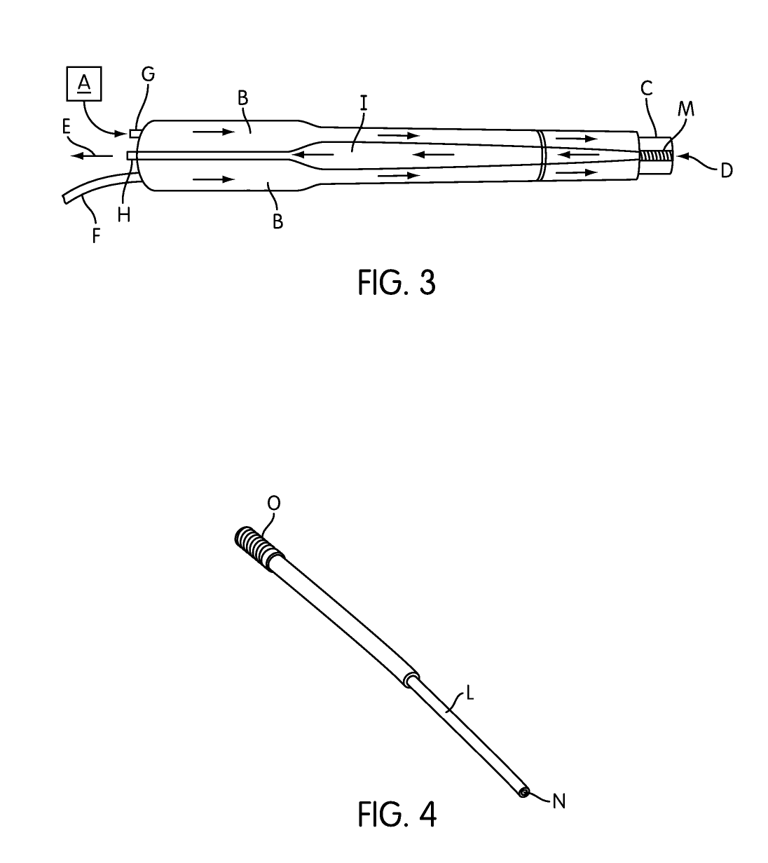

Devices should also include means for irrigating and aspirating tissues after fragmentation. This is illustrated in FIG. 3 which shows the hand held base unit of a device. In this figure, (A) represents a reservoir containing irrigation fluid and is connected to a port (G) leading into a channel (B). The channel runs to the distal end of the device where fluid exits and flows or sprays onto tissue. The end of the hand held base unit (C) attaches to the elongated rod (shown in FIG. 4) which vibrates at its tip in response to ultrasonic energy provided by an ultrasonic energy generator and transmitted via cord (F). The end of the base unit has an opening (N) that leads into a passageway (I in FIG. 3) extending from the tip of the rod to a port (H) at the opposite end of the support unit. Aspirated tissue exits this port in a stream (E) and may be delivered to a collection container. This container may contain fluids such as water or saline to preserve the tissue fragments and may optionally be chilled or contain fluid for fixing tissue. Samples recovered from the collection container may then be examined for histological features characteristic of disease or used in other diagnostic tests.

In order for the devices described above to provide information on the location of sampling sites, they should be integrated with existing systems for the stereotactic analysis of spatial arrangements. Many stereotactic systems have been developed (primarily to aid in neurosurgery) and can readily be adapted for the present invention. The first step in using these systems is to establish a three dimensional stereotactic coordinate system for reproducibly identifying positions in the tissue of the patient. This is usually accomplished using an apparatus or electrodes that are placed in fixed positions on the patient as a frame of reference. Diagnostic imaging procedures (e.g., CT scans or MRI scans) may then be performed to provide information concerning the internal tissues of the patient and the spatial relationship of the tissues to the established coordinate system. For example, imaging procedures may be used to provide information on the exact location of a tumor. After imaging, an important step is the registration step which takes place in the OR.

The final step is to use the medical devices described herein to obtain tissue fragments while, at the same time recording the exact position where each sample was collected. The sample from each site is retrieved from the device and diagnostically analyzed. In this way, pathologic differences in a tissue may be determined. For example, different sites from tissue containing a tumorous growth may provide information on areas in need of surgical resection and sections that can be spared. This is particularly important in tissues such as the brain where as much normal tissue as possible must be preserved.

### EXAMPLES

We collected brain tumor specimens using both, surgical forceps and CUSA and then performed histopathological and mass spectrometry analyses. The histopathology showed preservation of histology features required for diagnosis, and the direct mass spectrometry analysis of the tissue specimens using a DESI-LTQ instrument revealed molecular signatures indicative of neoplasia, as compared to specimens biopsied using surgical forceps. This new integrated surgical-sampling probe will enable the differentiation of tumor from non-tumor tissue based on measurements or imaging of the samples.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

1. A medical device comprising:
a) a hand held support;
b) an elongated metal rod extending from and attached to said hand-held support, wherein said rod terminates at its distal end in a tip that either itself vibrates in response to ultrasonic energy or comprises a separate component which vibrates in response to ultrasonic energy;
c) means for supplying ultrasonic energy to said tip or said separate component;
d) means for supplying irrigating fluid to said tip;
e) means for aspirating tissue fragments created at said tip;
f) means for stereotactically determining position of said tip.
g) means for collecting tissue samples.

2. The medical device of claim 1, wherein said means for stereotactically determining the position of the tip, comprises one or more electromagnetic emitters located on said device that transmit signals to a separate electromagnetic receiver or infrared energy detectors for optical tracking.

3. The medical device of claim 1 wherein said rod has a hollow core that provides a fluid passageway running along the length of said rod to said tip.

4. The medical device of claim 1, wherein said aspirating means creates suction in said hollow core of said rod to aspirate material through said tip in the direction of said hand held support.

5. The medical device of claim 4, wherein said fluid passageway terminates at its proximal end in a tissue collection container.

6. The medical device of claim 5, wherein said tissue collection container is chilled or comprises a fluid for fixing tissue samples.

7. The medical device of claim 1, wherein said means for supplying ultrasonic energy operates at a frequency of 15-100 kHz.

8. The medical device of claim 1, wherein said means for supplying irrigating fluid comprises a tubular channel running through said rod, wherein said channel is connected at its proximal end to a reservoir of irrigating fluid and wherein said channel terminates at the tip of said rod in an opening through which said irrigating fluid can flow.

9. The medical device of claim 1, wherein said hand held support comprises an actuator switch which, when activated, permits the transmission of ultrasonic energy to the tip of said rod or to said separate component which vibrates in response to ultrasonic energy and which does not permit said transmission when not activated.

10. The medical device of claim 9, wherein, said means for stereotactically determining the position of the distal end of said rod, comprises electromagnetic emitters located on said device that transmit signals to a separate electromagnetic receiver, wherein said electromagnetic receiver communicates signal data to a computer programmed to analyze position or infrared energy detectors for optical tracking.

11. A method of collecting a tissue sample from a biopsy site of a patient comprising:
a) inserting the tip of the medical device of claim 1 into said patient so that the distal end of said rod is positioned at said biopsy site;
b) transmitting ultrasonic energy to the tip of said rod or said separate component which vibrates in response to ultrasonic energy, thereby causing the fragmentation of tissue;
c) aspirating the fragments of tissue created in step b) into a collection container;
d) retrieving the fragments of tissue from said collection container.

12. A method of collecting from a patient a tissue sample that is mapped to a particular biopsy site, said method comprising:
a) establishing a three dimensional stereotactic coordinate system for reproducibly identifying positions in the tissue of said patient;
b) performing one or more diagnostic imaging procedures to identify areas where one or more biopsy samples will be taken;
c) collecting tissue samples using a medical device that:
i) fragments the tissue at specific sites;
ii) collects the fragments that have been generated;
iii) transmits a signal regarding its position in said stereotactic coordinate system, thereby providing a specific biopsy location.

13. The method of claim 12, wherein said medical device is the medical device of claim 1.

14. The method of claim 12, wherein, in step b) said diagnostic imaging procedures include a computed tomography (CT) scan.

15. The method of claim 12, wherein said diagnostic imaging procedures include a magnetic resonance imaging (MRI) scan.

16. The method of claim 15, wherein said is tissue obtained from the brain or breast of said patient.

17. The method of claim 16, wherein said medical device fragments tissues using ultrasonic vibrations.

18. The method of claim 17, wherein said tissue fragments are collected by aspiration.

19. The method of claim 18, wherein said medical device is the medical device of claim 1.

20. The method of claim 19, wherein at least one biopsy sample is obtained from a tumor.
